(19) United States
(12) Patent Application Publication
(10) Pub. No.: US 2014/0018885 A1
(43) Pub. Date: Jan. 16, 2014

(54) SYSTEMS AND METHODS FOR MAKING AND USING AN ELECTRICAL STIMULATION SYSTEM WITH A TISSUE-PENETRATING ELECTRODE

(71) Applicant: BOSTON SCIENTIFIC NEUROMODULATION CORPORATION, Valencia, CA (US)

(72) Inventor: Anne Margaret Pianca, Santa Monica, CA (US)

(21) Appl. No.: 13/939,900

(22) Filed: Jul. 11, 2013

Related U.S. Application Data
(60) Provisional application No. 61/670,989, filed on Jul. 12, 2012.

(51) Int. Cl. A61N 1/05

(52) U.S. Cl. CPC A61N 1/0551
USPC 607/72; 607/116; 607/117

(57) ABSTRACT

A lead assembly for providing electrical stimulation to a patient includes a penetrating electrode configured and arranged for stimulating patient tissue. The penetrating electrode has an outer surface and includes at least one sharpened tip configured and arranged to pierce patient tissue and anchor the lead assembly to the pierced patient tissue. An elongated, flexible tether has a first end and an opposing second end. The first end is coupled to the penetrating electrode. The second end is configured and arranged to couple to a pulse generator. An elongated conductor extends along the tether and is electrically coupled to the penetrating electrode.

### CROSS-REFERENCE TO RELATED APPLICATIONS

This application claims the benefit under 35 U.S.C. §119(e) of U.S. Provisional Patent Application Ser. No. 61/670,989 filed on Jul. 12, 2012, which is incorporated herein by reference.

### FIELD

The present invention is directed to the area of implantable electrical stimulation systems and methods of making and using the systems. The present invention is also directed to electrical stimulation systems having at least one electrode configured and arranged for penetrating patient tissue, as well as methods of making and using the tissue-penetrating electrodes and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

Dorsal root ganglia are nodules of cell bodies disposed along the dorsal roots of spinal nerves. Dorsal root ganglia are disposed external to the epidural space. Dorsal root ganglia, however, are disposed in proximity to the spinal cord and the vertebral column.

### BRIEF SUMMARY

In one embodiment, an implantable lead assembly for providing electrical stimulation to a patient includes at least one first penetrating electrode configured and arranged for stimulating patient tissue. The at least one first penetrating electrode has an outer surface and includes at least one sharpened tip configured and arranged to pierce patient tissue and anchor the lead assembly to the pierced patient tissue. An elongated, flexible first tether has a first end and an opposing second end. The first end is coupled to the at least one first penetrating electrode. The second end is configured and arranged to couple to a pulse generator. An elongated first conductor extends along the first tether and electrically couples to the at least one first penetrating electrode.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of another embodiment of an electrical stimulation system that includes a percutaneous lead body coupled to a control module, according to the invention;

FIG. 2A is a schematic view of one embodiment of a plurality of connector assemblies disposed in the control module of FIG. 1, the connector assemblies configured and arranged to receive the proximal portions of the lead bodies of FIG. 1, according to the invention;

FIG. 2B is a schematic view of one embodiment of a proximal portion of the lead body of FIG. 1, a lead extension, and the control module of FIG. 1, the lead extension configured and arranged to couple the lead body to the control module, according to the invention;

FIG. 3A is a schematic transverse cross-sectional view of spinal nerves extending from a spinal cord, the spinal nerves including dorsal root ganglia;

FIG. 3B is a schematic perspective view of a portion of the spinal cord of FIG. 3A disposed in a portion of a vertebral column with the dorsal root ganglia of FIG. 3A extending outward from the vertebral column;

FIG. 3C is a schematic top view of a portion of the spinal cord of FIG. 3A disposed in a vertebral foramen defined in a vertebra of the vertebral column of FIG. 3B, the vertebra also defining intervertebral foramina extending between an outer surface of the vertebra and the vertebral foramen, the intervertebral foramina providing an opening through which the dorsal root ganglia of FIG. 3B can extend outward from the spinal cord of FIG. 3B;

FIG. 3D is a schematic side view of two vertebrae of the vertebral column of FIG. 3B, the vertebrae defining an intervertebral foramen through which one of the dorsal root ganglia of FIG. 3B can extend outward from the spinal cord of FIG. 3B;

FIG. 4 is a schematic view of several different embodiments of an implantable electrical stimulation system that includes a lead assembly coupleable to a pulse generator, the lead assembly including a penetrating electrode coupled to a tether, according to the invention;

FIG. 5 is a schematic view of one embodiment of the lead assembly of FIG. 4 that includes a lead body coupled to the tether of FIG. 4, where the lead body is configured and arranged to couple to the pulse generator of FIG. 4, according to the invention;

FIG. 6A is a schematic side view of one embodiment of the penetrating electrode of FIG. 4 piercing a dorsal root ganglion, according to the invention;

FIG. 6B is a schematic side view of another embodiment of the penetrating electrode of FIG. 4 piercing the dorsal root ganglion of FIG. 4, according to the invention;

FIG. 7A is a schematic side view of another embodiment of the penetrating electrode of FIG. 4, the penetrating electrode having a plurality of bends, according to the invention;

FIG. 7B is a schematic side view of yet another embodiment of the penetrating electrode of FIG. 4, the penetrating electrodes having a plurality of sharpened tips oriented in opposite directions, according to the invention;

FIG. 7C is a schematic side view of another embodiment of the penetrating electrode of FIG. 4, the penetrating electrode having a plurality of sharpened tips oriented in the same direction, according to the invention;

FIG. 8 is a schematic view of another embodiment of the lead assembly of FIG. 4, the lead assembly including a plurality of penetrating electrodes coupled to a single tether, according to the invention;

FIG. 9 is a schematic view of yet another embodiment of the lead assembly of FIG. 4, the lead assembly including a plurality of penetrating electrodes coupled to a plurality of tethers, according to the invention;

FIG. 10A is a schematic perspective view of the spinal cord of FIG. 3A disposed along a longitudinal transverse view of a portion of the vertebral column of FIG. 3B, where a side view of one embodiment of a distal portion of a needle is shown inserted into an epidural space between the spinal cord and the vertebral column from a location external to the vertebral column, and where the penetrating electrode and tether of FIG. 4 are extended from the distal portion of the needle, through an intervertebral foramen, and anchored to a dorsal root ganglion, according to the invention;

FIG. 10B is a schematic perspective view of the spinal cord of FIG. 3A disposed along a longitudinal transverse view of a portion of the vertebral column of FIG. 3B, where the penetrating electrode and tether of FIG. 4 are extended into an epidural space, through an intervertebral foramen, and anchored to a dorsal root ganglion, according to the invention;

FIG. 11A is a schematic perspective view illustrating the insertion of a first introducer of a series of introducer to obtain access to a dorsal root ganglion for implantation of the lead assembly of FIG. 4, according to the invention;

FIG. 11B is a schematic perspective view illustrating the insertion of a second introducer of a series of introducer to obtain access to a dorsal root ganglion for implantation of the lead assembly of FIG. 4, according to the invention;

FIG. 11C is a schematic perspective view illustrating the insertion of a third introducer of a series of introducer to obtain access to a dorsal root ganglion for implantation of the lead assembly of FIG. 4, according to the invention;

FIG. 11D is a schematic perspective view illustrating the insertion of a fourth introducer of a series of introducer to obtain access to a dorsal root ganglion for implantation of the lead assembly of FIG. 4, according to the invention;

FIG. 11E is a schematic perspective view illustrating the removal of the first, second and third introducers of the series of introducer and the implantation of the lead assembly of FIG. 4 within a dorsal root ganglion, according to the invention; and

FIG. 12 is a schematic overview of one embodiment of components of an electrical stimulation system, according to the invention.

### DETAILED DESCRIPTION

The present invention is directed to the area of implantable electrical stimulation systems and methods of making and using the systems. The present invention is also directed to electrical stimulation systems having at least one electrode configured and arranged for penetrating patient tissue, as well as methods of making and using the tissue-penetrating electrodes and electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, deep brain stimulation leads, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,672,734; 7,761,165; 7,949,395; 7,974,706; 8,175,710; 8,224,450; and 8,364,278; and U.S. Patent Application Publication No. 2007/0150036, all of which are incorporated by reference.

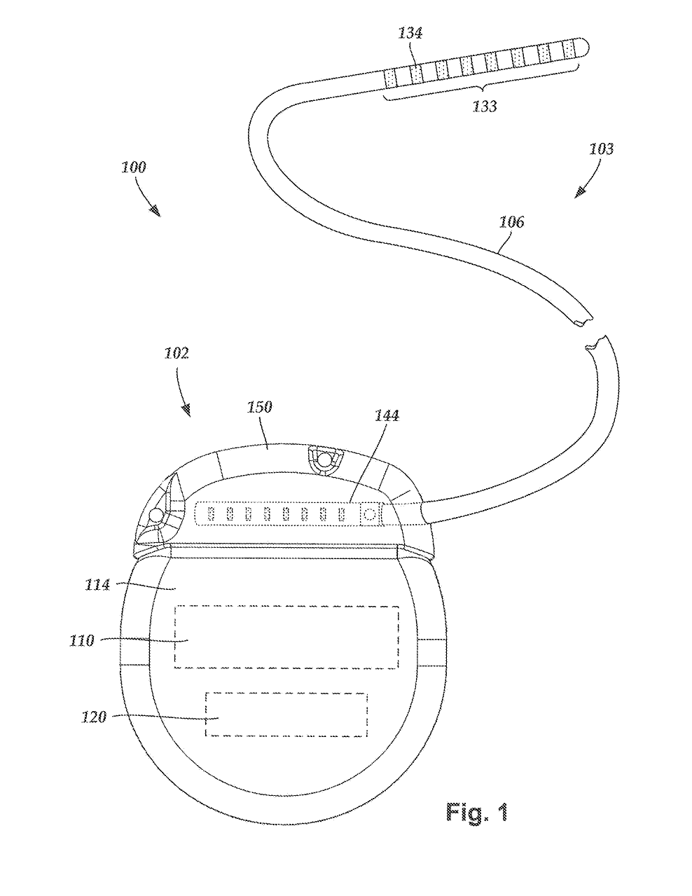

FIG. 1 illustrates schematically one embodiment of an electrical stimulation system 100. The electrical stimulation system 100 includes a control module (e.g., a stimulator or pulse generator) 102 and a percutaneous lead 103. The lead 103 includes a plurality of electrodes 134 that form an array of electrodes 133. The control module 102 typically includes an electronic subassembly 110 and an optional power source 120 disposed in a sealed housing 114. The lead 103 includes a lead body 106 coupling the control module 102 to the plurality of electrodes 134. In at least some embodiments, the lead body 106 is isodiametric.

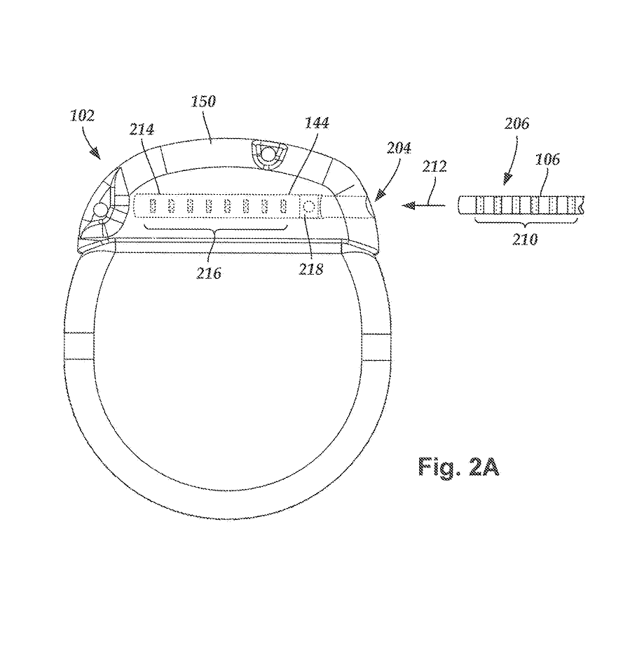

The control module 102 typically includes one or more connector assemblies 144 into which the proximal end of the lead body 106 can be plugged to make an electrical connection via connector contacts (e.g., 216 in FIG. 2A) disposed in the connector assembly 144 and terminals (e.g., 210 in FIG. 2A) disposed along the lead body 106. The connector contacts are coupled to the electronic subassembly 110 and the terminals are coupled to the electrodes 134. Optionally, the control module 102 may include a plurality of connector assemblies 144.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 224 (see FIG. 2C) can be disposed between the lead body 106 and the control module 102 to extend the distance between the lead body 106 and the control module 102.

The electrical stimulation system or components of the electrical stimulation system, including the lead body 106 and the control module 102, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, titanium, or rhenium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the lead body 106 are typically disposed in, or separated by, a non-conductive, biocompatible material including, for example, silicone, polyurethane, and the like or combinations thereof. The lead body 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead body 106 to the proximal end of the lead body 106.

Terminals (e.g., 210 in FIG. 2A) are typically disposed at the proximal end of the lead body 106 for connection to corresponding conductive contacts (e.g., 216 in FIG. 2A) in one or more connector assemblies (e.g., 144 in FIG. 1) disposed on, for example, the control module 102 (or to other devices, such as conductive contacts on a lead extension, an operating room cable, a splitter, an adaptor, or the like).

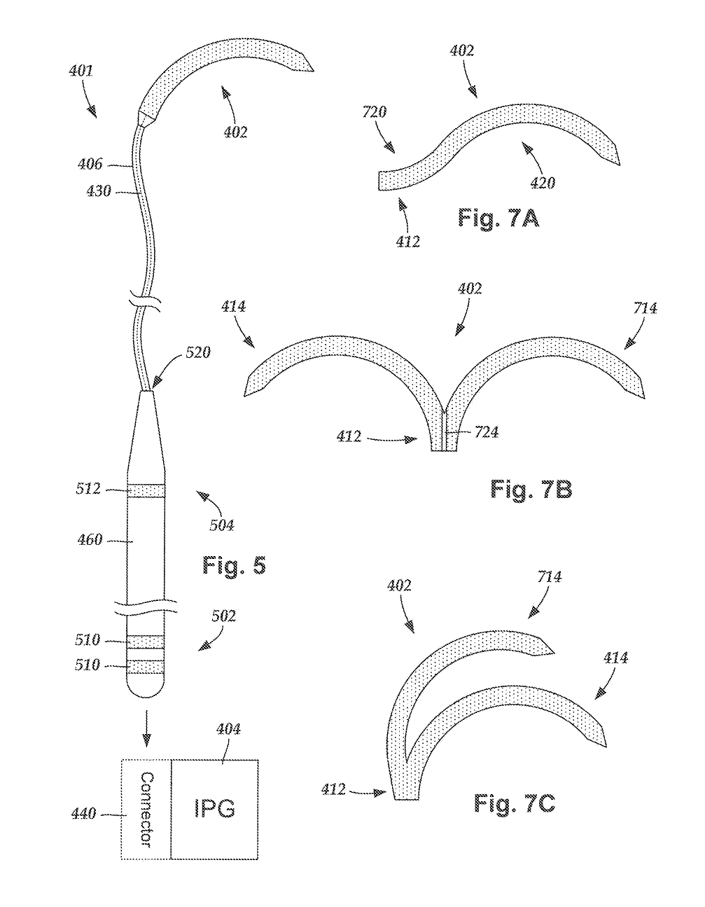

Conductive wires (see e.g., 508 of FIG. 5B) extend from the plurality of terminals (see e.g., 210 in FIG. 2A) to the plurality of electrodes 133. Typically, each of the plurality of terminals is electrically coupled to at least one of the plurality of electrodes 133. In some embodiments, each of the plurality of terminals is coupled to a single electrode 134 of the plurality of electrodes 133.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the lead 103. The one or more lumens may, optionally, be flushed continually, or on a regular basis, with saline, epidural fluid, or the like. The one or more lumens can be permanently or removably sealable at the distal end.

As discussed above, the lead body 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, the lead body 106 includes eight terminals that are shown coupled with eight conductive contacts disposed in the connector assembly 144.

FIG. 2A is a schematic side view of one embodiment of a connector assembly 144 disposed on the control module 102. In FIG. 2A, the proximal end 306 of the lead body 106 is shown configured and arranged for insertion to the control module 102.

In FIG. 2A, the connector assembly 144 is disposed in the header 150. In at least some embodiments, the header 150 defines a port 204 into which the proximal end 206 of the lead body 106 with terminals 210 can be inserted, as shown by directional arrows 212, in order to gain access to the connector contacts disposed in the connector assembly 144.

The connector assembly 144 includes a connector housing 214 and a plurality of connector contacts 216 disposed therein. Typically, the connector housing 214 defines a port (not shown) that provides access to the plurality of connector contacts 216. In at least some embodiments, the connector assembly 144 further includes a retaining element 218 configured and arranged to fasten the corresponding lead body 106 to the connector assembly 144 when the lead body 106 is inserted into the connector assembly 144 to prevent undesired detachment of the lead body 106 from the connector assembly 144. For example, the retaining element 218 may include an aperture 220 through which a fastener (e.g., a set screw, pin, or the like) may be inserted and secured against an inserted lead body 106.

When the lead body 106 is inserted into the port 204, the connector contacts 216 can be aligned with the terminals 210 disposed on the lead body 106 to electrically couple the control module 102 to the electrodes (134 of FIG. 1) disposed at a distal end of the lead body 106. Examples of connector assemblies in control modules are found in, for example, U.S. Pat. Nos. 7,244,150 and 8,224,450, which are incorporated by reference.

In at least some embodiments, the electrical stimulation system includes one or more lead extensions. The lead body 106 can be coupled to one or more lead extensions which, in turn, are coupled to the control module 102. In FIG. 2B, a lead extension connector assembly 222 is disposed on a lead extension 224. The lead extension connector assembly 222 is shown disposed at a distal end 226 of the lead extension 224. The lead extension connector assembly 222 includes a contact housing 228. The contact housing 228 defines at least one port 230 into which a proximal end 206 of the lead body 106 with terminals 210 can be inserted, as shown by directional arrow 238. The lead extension connector assembly 222 also includes a plurality of connector contacts 240. When the lead body 106 is inserted into the port 230, the connector contacts 240 disposed in the contact housing 228 can be aligned with the terminals 210 on the lead body 106 to electrically couple the lead extension 224 to the electrodes (134 of FIG. 1) disposed at a distal end (not shown) of the lead body 106.

The proximal end of a lead extension can be similarly configured and arranged as a proximal end of a lead body. The lead extension 224 may include a plurality of conductive wires (not shown) that electrically couple the connector contacts 240 to terminal on a proximal end 248 of the lead extension 224. The conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a lead extension connector assembly disposed in another lead extension. In other embodiments (as shown in FIG. 2B), the proximal end 248 of the lead extension 224 is configured and arranged for insertion into the connector assembly 144 disposed on the control module 102.

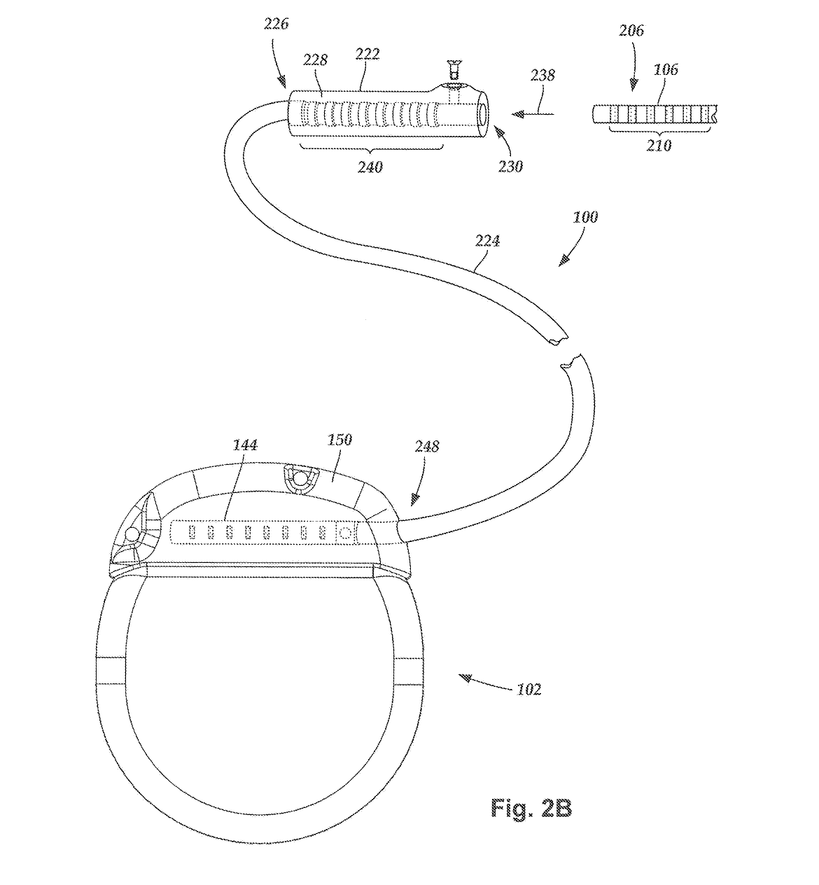

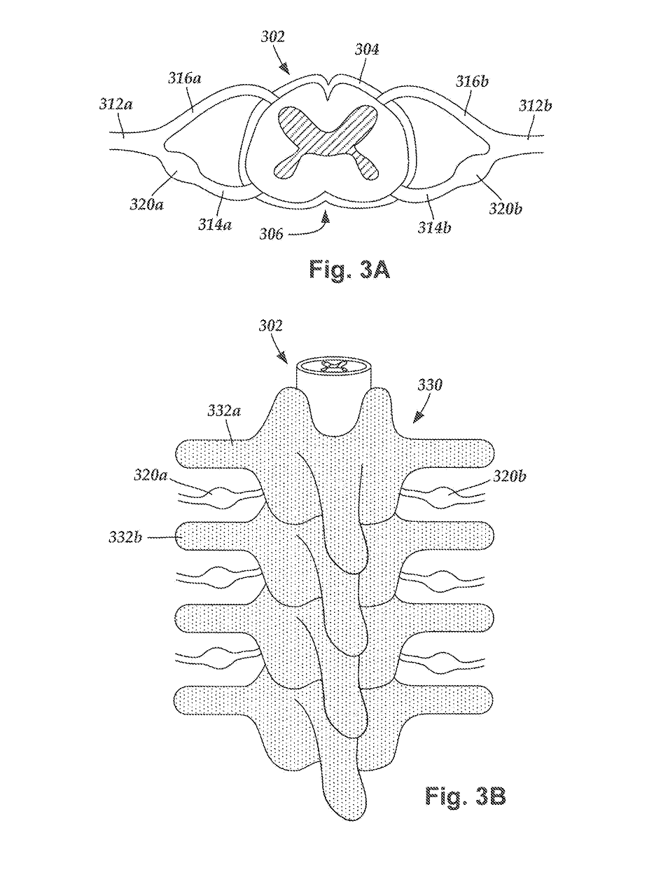

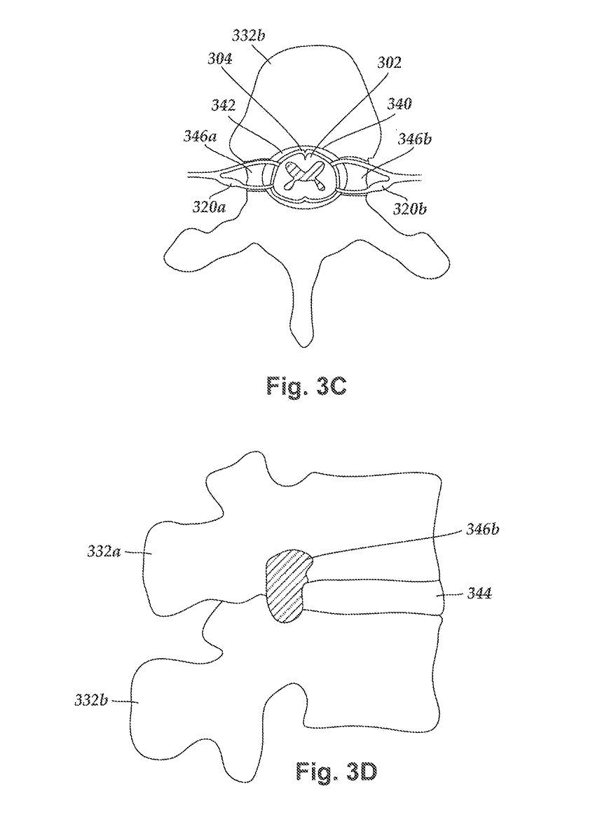

Turning to FIG. 3A, in at least some embodiments one or more dorsal root ganglia (“DRG”) are potential target stimulation locations. FIG. 3A schematically illustrates a transverse cross-sectional view of a spinal cord 302 surrounded by dura 304. The spinal cord 302 includes a midline 306 and a plurality of levels from which spinal nerves 312a and 312b extend. In at least some spinal cord levels, the spinal nerves 312a and 312b extend bilaterally from the midline 306 of the spinal cord 302. In FIG. 3A, the spinal nerves 312a and 312b are shown attaching to the spinal cord 302 at a particular spinal cord level via corresponding dorsal roots 314a and 314b and corresponding ventral (or anterior) roots 316a and 316b. Typically, the dorsal roots 314a and 314b relay sensory information into the spinal cord 302 and the ventral roots 316a and 316b relay motor information outward from the spinal cord 302. The DRG 320a and 320b are nodules of cell bodies that are disposed along the dorsal roots 316a and 316b in proximity to the spinal cord 302.

FIG. 3B schematically illustrates a perspective view of a portion of the spinal cord 302 disposed along a portion of a vertebral column 330. The vertebral column 330 includes stacked vertebrae, such as vertebrae 332a and 332b, and a plurality of DRGs 320a and 320b extending outwardly bilaterally from the spinal cord 302 at different spinal cord levels.

FIG. 3C schematically illustrates a top view of a portion of the spinal cord 302 and surrounding dura 304 disposed in a vertebral foramen 340 defined in the vertebra 332b. The vertebrae, such as the vertebrae 332a and 332b, are stacked together and the vertebral foramina 340 of the vertebrae collectively form a spinal canal through which the spinal cord 302 extends. The space within the spinal canal between the dura 304 and the walls of the vertebral foramen 340 defines the epidural space 342. Intervertebral foramina 346a and 346b, defined bilaterally along sides of the vertebra 332b, form openings through the vertebra 332b between the epidural space 342 and the environment external to the vertebra 332b.

FIG. 3D schematically illustrates a side view of two vertebrae 332a and 332b coupled to one another by a disc 344. In FIG. 3D, the intervertebral foramen 346b is shown defined between the vertebrae 332a and 332b. The intervertebral foramen 346b provides an opening for one or more of the dorsal root 314b, ventral root 316b, and DRG 320b to extend outwardly from the spinal cord 302 to the environment external to the vertebrae 332a and 332b.

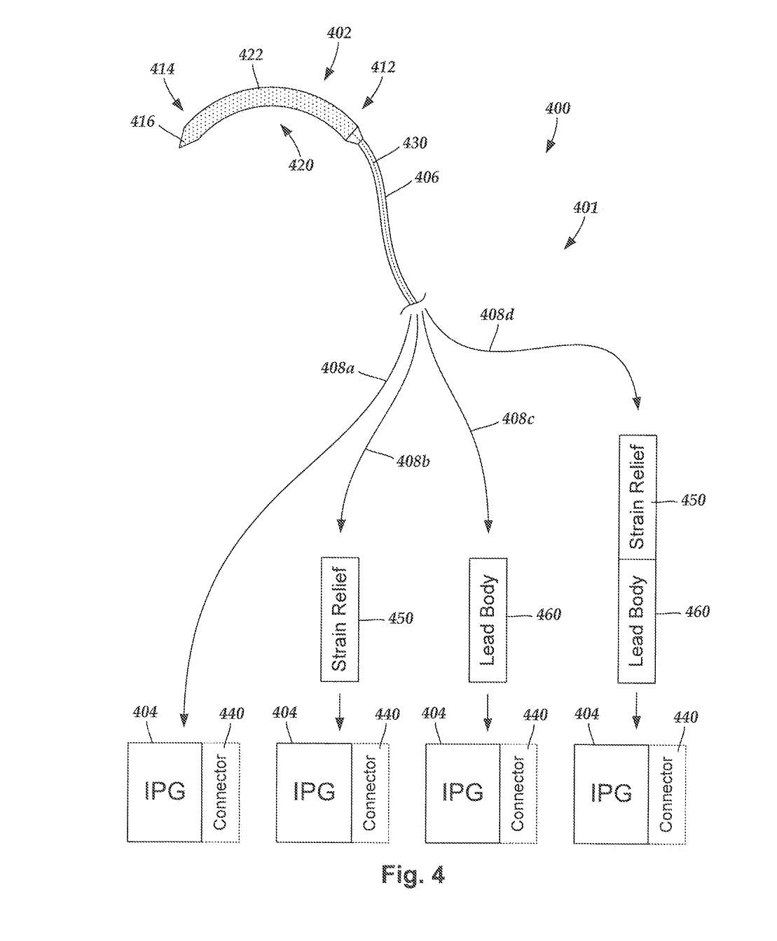

Turning to FIG. 4, electrode placement at a target stimulation location can be important for obtaining efficacious patient response to stimulation. Sometimes an electrode-containing portion (e.g., a distal end) of a lead may migrate from an intended treatment site over time due to patient movement. When the electrode-containing portion of a lead migrates far enough away from the target stimulation location, a loss of efficacy may occur and surgical re-implantation may become necessary to re-establish efficacy. One way to reduce migration is to anchor one or more portions of the lead to patient tissue in proximity to the target stimulation location.

As herein described, an electrical stimulation system includes one or more electrodes configured and arranged to penetrate patient tissue (“penetrating electrodes”). The penetrating electrodes are configured and arranged for anchoring to patient tissue and providing stimulation at the location of the anchoring. In some embodiments, the one or more penetrating electrodes can be anchored near to (i.e., in operational proximity to) a target stimulation location. In other embodiments, the one or more penetrating electrodes can be anchored to the target stimulation location itself For example, in at least some embodiments the target stimulation location is a DRG. In which case, the one or more penetrating electrodes can be anchored to patient tissue either in operational proximity to the DRG, or to the DRG itself In the latter case, the one or more penetrating electrodes can be anchored to the DRG such that the one or more penetrating electrodes are disposed partially, or even completely, within the DRG during operation.

FIG. 4 is a schematic view of several different embodiments of an implantable electrical stimulation system 400. The electrical stimulation system 400 includes a lead assembly 401 and an implantable pulse generator 404. The lead assembly 401 includes a penetrating electrode 402 and a tether 406 that is coupled to the penetrating electrode 402 and that is configured and arranged to couple to the implantable pulse generator 404.

The tether 406 is coupleable to the pulse generator 404 in any suitable manner. In FIG. 4, four alternate lead assemblies 401 are shown, as illustrated by arrows 408a-408d. Each of the different lead assembly 401 embodiments includes the penetrating electrode 402 and the tether 406 and is discussed in more detail below. It will be understood that the four different alternate embodiments of the lead assembly shown in FIG. 4 are exemplary and are not meant to be limiting.

The penetrating electrode 402 has a proximal end 412 and an opposing distal end 414. The distal end 414 includes a sharpened tip 416 configured and arranged to pierce patient tissue. Pierceable patient tissue includes, for example, nervous tissue, connective tissue, epithelial tissue, muscle tissue, one or more tissues forming the DRG, or the like. It will be understood that the patient tissues which the penetrating electrode is configured and arranged to penetrate, or pierce, is not meant to be limited to fluid-dominant tissues, such as blood, lymph fluid, or the like.

The penetrating electrode 402 can have any shape suitable for piercing patient tissue and anchoring to the pierced tissue (see e.g., FIGS. 4 and 7A-7C). In at least some embodiments, the penetrating electrode 402 has a shape that is similar to a suture needle. In at least some embodiments, the penetrating electrode 402 includes a single bend. In alternate embodiments, the penetrating electrode 402 includes either no bends, or multiple bends. In FIG. 4, the penetrating electrode 402 is shown having a first bend 420. Designing the penetrating electrode 402 with the first bend 420 may facilitate maneuvering of the penetrating electrode 402 within confined spaces, including, for example, piercing of patient tissue in situ. In at least some embodiments, the curvature of the first bend 420 is no greater than 180°, 150°, 120°, 90°, 60°, 30°, or less. In at least some embodiments, the curvature of the first bend 420 is at least 5°, 15°, 25°, 35°, 45°, 55°, 65°, 75°, or more.

It will be understood that the relative dimensions of the penetrating electrode shown in FIG. 4, as well as in subsequent FIGS. 5A-10B and 11E, are exemplary and are meant for illustrative purposes only. The penetrating electrode can have any suitable length or width. Additionally, the width can be either larger or smaller for a given length from what is shown in the figures.

In at least some embodiments, the penetrating electrode has a length of at least 3 mm. In at least some embodiments, the penetrating electrode has a length that is no greater than 10 mm. In at least some embodiments, the penetrating electrode has a width of at least 0.3 mm. In at least some embodiments, the penetrating electrode has a width that is no greater than 1.5 mm.

It may be desirable to increase the area of an outer surface 422 of the penetrating electrode 402 to reduce charge density during stimulation. The surface area of the penetrating electrode 402 can be increased in any suitable manner including, adding one or more mechanical features, applying one or more coatings, or the like or combinations thereof. Examples of suitable techniques for adding mechanical features to increase the surface area of the penetrating electrode 402 include roughening, grit blasting, knurling, notching, or the like or combinations thereof. Examples of suitable coatings for applying to the penetrating electrode 402 to increase the surface area of the penetrating electrode 402 include coatings of iridium oxide, titanium nitride, platinum black, platinum gray, or the like or combinations thereof.

The tether 406 is flexible to facilitate anchoring of the penetrating electrode 402 to patient tissue. In at least some embodiments, the tether 406 is formed from a lightweight material. In at least some embodiments, the tether 406 is formed from an elastic material. The tether 406 can have any suitable outer diameter. The tether 406 can have an outer diameter that is greater than, equal to, or less than a width of the penetrating electrode 402. In at least some embodiments, the tether 406 has an outer diameter that is no greater than 0.03 inches (approximately 0.08 cm), 0.025 inches (approximately 0.06 cm), 0.02 inches (approximately 0.05 cm), 0.015 inches (approximately 0.04 cm), 0.01 inches (approximately 0.03 cm), or less. In at least some embodiments, the tether has a length of at least 10 cm. In at least some embodiments, the tether has a length that is no greater than 100 cm.

An elongated conductor 430 extends along the tether 406 and electrically couples the penetrating electrode 402 to the implantable pulse generator 404. In at least some embodiments, the conductor 430 includes slack to facilitate expansion of a length of the tether 406. The slack in the conductor 430 may include one or more overlapping regions, such as a coiled configuration. Additionally or alternately, the slack in the conductor 430 may include a bellows configuration, or the like. The conductor 430 can be single filar, multi filar, one or more cables, or the like. The conductor 430 can, optionally, be formed from multiple conductors. In which case, the one or more conductors 430 may each be individually encased in one or more layers of conductor insulation.

Optionally, one or more layers of tether insulation are disposed around the one or more elongated conductors 430 along a longitudinal length of the tether 406. In embodiments where the multiple conductors 430 are individually encased in conductor insulation, the tether insulation is disposed around the conductor insulation such that the tether insulation collectively encases each of the multiple conductors 430 and their respective conductor insulations. Suitable tether insulation materials include, for example, polyurethane, polytetrafluoroethylene, ethylene tetrafluoroethylene, silicone, or the like or combinations thereof.

Optionally, one or more coatings are applied to an outer surface of the tether 406. In at least some embodiments, a lubricious coating is applied to the outer surface of the tether 406 to facilitate maneuvering of the tether 406 in tight spaces. In at least some embodiments, a hydrophilic coating is applied to the outer surface of the tether 406 to further facilitate maneuvering of the tether 406 in tight spaces and to modulate interactions with adjacent tissue.

In at least some embodiments (as represented by the arrow 408a in FIG. 4), the tether 406 is coupled directly to the pulse generator 404. In some embodiments, the tether 406 is wired directly to the pulse generator 404. In other embodiments, the tether 406 is removably coupleable to the pulse generator 404 via an optional connector 440 disposed on or in the pulse generator 404.

The pulse generator 404 can be implemented in any suitable manner. For example, the pulse generator 404 can be disposed in a control module (see e.g., 102 in FIG. 1). Alternately, the pulse generator 404 can be disposed in a microstimulator. Examples of microstimulators are found in, for example, U.S. Pat. No. 8,204,595, which is incorporated by reference.

Optionally, the lead assembly 401 includes a strain relief 450 to modulate the effects of bending of one or more portions of the tether 406. Examples of strain reliefs are found in, for example, U.S. Patent Applications Publication Nos. 2012/0316615; 2013/0105071; and U.S. patent application Ser. No. 13/750,725, each of which is incorporated herein by reference in its entirety.

In at least some embodiments (as represented by the arrow 408b in FIG. 4), the tether 406 is coupleable to the pulse generator 404 by the optional strain relief 450. In at least some embodiments, the strain relief 450 reduces the likelihood of an electrical decoupling of the tether 406 from the pulse generator 404 during, for example, patient movement. In at least some embodiments, when the penetrating electrode 402 is coupled to patient tissue the strain relief 450 reduces the likelihood of decoupling of the penetrating electrode 402 from the patient tissue to which the penetrating electrode 402 is coupled during, for example, patient movement.

Optionally, the lead assembly 401 includes a lead body 460 (see e.g., FIG. 5). In at least some embodiments (as represented by the arrows 408c and 408d in FIG. 4), the tether 406 is coupled to the pulse generator 404 by the optional lead body 460 (discussed in more detail below, with reference to FIG. 5). In at least some embodiments, the lead body 460 may include a strain relief 450 (as represented by the arrow 408d in FIG. 4). In embodiments of the lead assembly 401 that include the lead body 460, the lead body 460 is coupleable to the pulse generator 404, either directly or via the connector 440.

In embodiments of the lead assembly 401 that include the lead body 460, the lead body 406 can be designed in any suitable manner. FIG. 5 is a schematic view of one embodiment of the lead body 460 coupled to the tether 460 and configured and arranged to couple to the pulse generator 404. The lead body 460 has a proximal end 502 and a distal end 504. The proximal end 502 of the lead body 460 is configured and arranged to couple to the pulse generator 404. In at least some embodiments, the tether conductor 430 is coupled to one or more terminals 510 disposed along the proximal end 502 of the lead body 460. In which case, the one or more terminals 510 are configured and arranged for coupling electrically with the pulse generator 404.

Optionally, one or more lead electrodes 512 are disposed along the distal end 504 of the lead body 460 and are electrically coupled to the one or more terminals 510. In which case, in at least some embodiments the penetrating electrode 402 and one of the lead electrodes 512 may operate as an anode/cathode pair.

In FIG. 5, the tether 406 is shown extending outwardly from the distal end 504 of the lead body 460. In at least some embodiments, the distal end 460 of the lead body 460 defines an aperture 520 through which the penetrating electrode 402, the tether 406, or both, can extend. In alternate embodiments, the tether 406 extends from another portion of the lead body 460, such as the proximal end 502 of the lead body 460, or an intermediate portion of the lead body 460. It will be understood that the penetrating electrode 402 may be smaller as compared to the lead body 460 than what is shown in FIG. 5.

In at least some embodiments, the lead body 460 has a diameter that is larger than a diameter of the tether 406. In at least some embodiments, the penetrating electrode 402 is disposed in the lead body 460 during implantation of the lead body 460. In at least some embodiments, an entire length of the tether 406 is disposed in the lead body 460 during implantation of the lead body 460 into the patient.

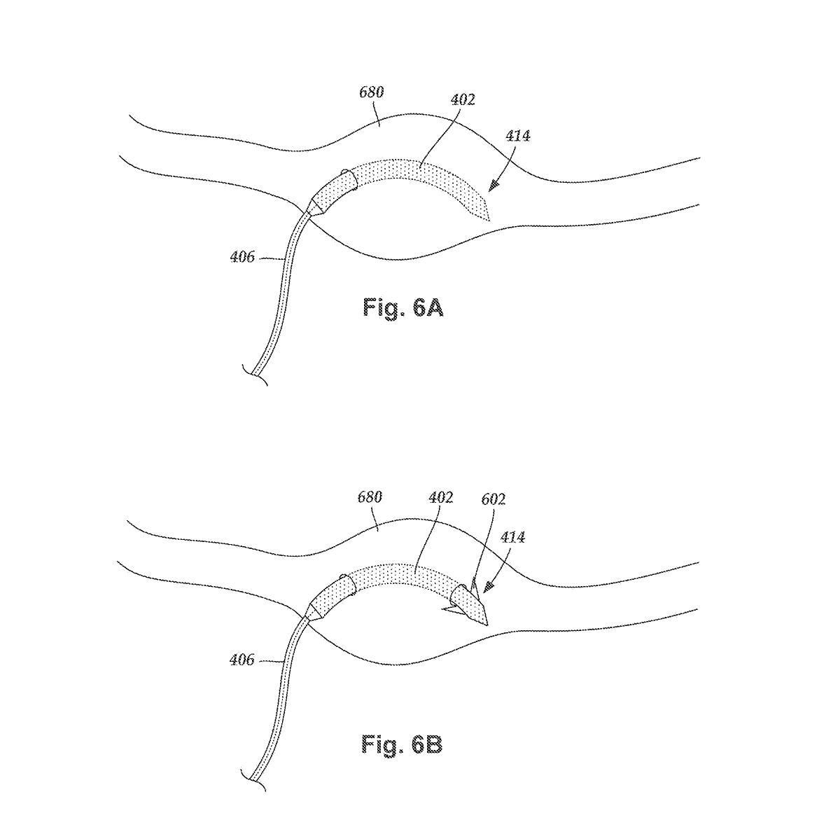

Turning to FIGS. 6A-6B, in at least some embodiments the one or more penetrating electrodes are configured and arranged to anchor to patient tissue at, or in operational proximity to, the target stimulation location. One potential target stimulation location is one or more or the patient's dorsal root ganglia (“DRG”).

FIGS. 6A-6B are schematic side views of two different embodiments of the penetrating electrode 402 anchored to a DRG 680. In FIG. 6A, the penetrating electrode 402 is anchored to the DRG 680 such that the distal end 414 of the penetrating electrode 402 is disposed within the DRG 680. The penetrating electrode 402 can be disposed either partially, or entirely, within the DRG 680. In FIG. 6B, the penetrating electrode 402 is anchored to the DRG 680 such that the distal end 414 of the penetrating electrode 402 is extended into and back out of the DRG 680.

In other embodiments where the DRG 680 is the target stimulation location, the penetrating electrode 402 can be anchored to other patient tissue in proximity to the DRG 680. In yet other embodiments where the DRG 680 is the target stimulation location, the penetrating electrode 402 can be anchored to both the DRG 680 and to other patient tissue in proximity to the DRG 680.

Piercing patient tissue with a tissue-penetrating electrode may provide advantages over other electrode-placement techniques. For example, when the penetrating electrode 402 is disposed at least partially within the target stimulation location, stimulation may be efficacious at amplitudes that may otherwise be sub-therapeutic were the penetrating electrode to be disposed external to the target stimulation location. Moreover, stimulating a target stimulation location from a position within the target stimulation location itself may increase the selectivity of the stimulation because the stimulation energy directly contacts the target stimulation location and, therefore, propagates through the target stimulation location prior to reaching non-target locations. In which case, the stimulation energy may be greatly diminished before reaching any non-target stimulation locations.

In at least some embodiments, the one or more penetrating electrodes 402 are configured and arranged to anchor to patient tissue such that the penetrating electrode 402 does not move relative to the DRG 680. In FIG. 6B, the penetrating electrode 402 is shown as, optionally, including a retention member (e.g., one or more barbs, or the like) 602. When the penetrating electrode 402 is implanted and is piercing the target stimulation location (e.g., the DRG 680), it may be advantageous to design the penetrating electrode 402 to include the retention member 602 to prevent the penetrating electrode 402 from slipping out of the target stimulation location, and potentially migrating away from the target stimulation location.

In at least some embodiments, the penetrating electrode 402 is configured and arranged to mitigate trauma associated with piercing of the target stimulation location (e.g., the DRG 680). In at least some embodiments, the penetrating electrode 402 is coated with a pharmacological agent (e.g., one or more steroids, antibiotics, or the like or combinations thereof), for reducing one or more ill-effects to the patient (e.g., inflammation, infection, scarring, or the like or combinations thereof) caused by piercing of the DRG 680.

Stimulation energy typically propagates through patient tissue more readily when the patient tissue is not scarred. Thus, a further advantage of coating the penetrating electrode 402 with one or more pharmacological agents for reducing scarring is that reducing scarring may, consequently, reduce the amplitude of stimulation energy needed to provide therapy to the patient and provide a more controlled effect.

Turning to FIG. 7A, the penetrating electrode can be any shape suitable for piercing patient tissue. FIG. 7A is a schematic side view of another embodiment of a penetrating electrode 402 having multiple bends. In FIG. 7A, the penetrating electrode 402 includes the first bend 420 and a second bend 720 in series with the first bend 420 along a length of the penetrating electrode 402. Using multiple bends may improve the anchoring ability of the penetrating electrode 402. Using multiple bends may also facilitate fine-tuning of the placement of the penetrating electrode at a particular position within the target stimulation location.

In at least some embodiments, the curvature of the second bend 720 is no greater than 180°, 150°, 120°, 90°, 60°, 30°, or less. In at least some embodiments, the curvature of the second bend 720 is at least 5°, 15°, 25°, 35°, 45°, 55°, 65°, 75°, or more. In at least some embodiments, the curvature of the second bend 720 extends opposite to the curvature of the first bend 420 such that the first bend 420 and the second bend 720 collectively from an S-shape. It will be understood that the penetrating electrode can be formed into many other shapes including, for example, straight, corkscrew (i.e., helical), or the like. The first bend 420 can have a curvature that is less than, equal to, or greater than the curvature of the second bend 720.

Turning to FIGS. 7B-7C, in at least some embodiments the penetrating electrode includes multiple distal tips. FIGS. 7B-7C are schematic side views of several different embodiments of a penetrating electrode 402 having multiple distal ends 414 and 714. In FIG. 7B, the distal ends 414 and 714 are oriented in opposite directions from one another. In FIG. 7C, the distal ends 414 and 714 are oriented in substantially the same direction as one another. It will be understood that the penetrating electrode 402 can have any suitable number of distal ends including, for example, one, two, three, four, five, six, seven, eight, nine, ten, or more distal ends.

Any number of the distal ends can be sharpened to facilitate piercing of patient tissue on or around the target stimulation location. In FIGS. 7B-7C, the distal ends 414 and 714 are both sharpened so that each of the distal ends 414 and 714 can pierce patient tissue independently from one another. It may be advantageous to provide a plurality of sharpened distal ends so that the distal ends can pierce the target stimulation location in multiple different places, thereby enabling stimulation energy to be directed to multiple different locations within or around the target stimulation location. This may further be advantageous when, for example, the target stimulation location includes multiple parts (e.g., a multi-lobed DRG, or the like), or when multiple target stimulation locations are grouped in proximity to one another, or when the target stimulation location is relatively large compared to the size of the penetrating electrode.

In FIGS. 7B-7C, the penetrating electrodes each include a single proximal end 412. It may be an advantage to design the penetrating electrode 402 with a plurality of distal ends and a single proximal end. Such a configuration enables the penetrating electrode 402 to couple to a single tether 406 (and, in at least some cases a single conductor 430 within the tether 406), while potentially expanding the stimulation coverage as compared to the penetrating electrode of FIG. 4 without increasing the amplitude of stimulation. Optionally, an insulator 724 electrically isolates the distal ends of the penetrating electrode from one another. In which case, each of the distal ends of the penetrating electrode can be operated independently from one another using different conductors 430 coupled to the penetrating electrodes on either side of the insulator 724.

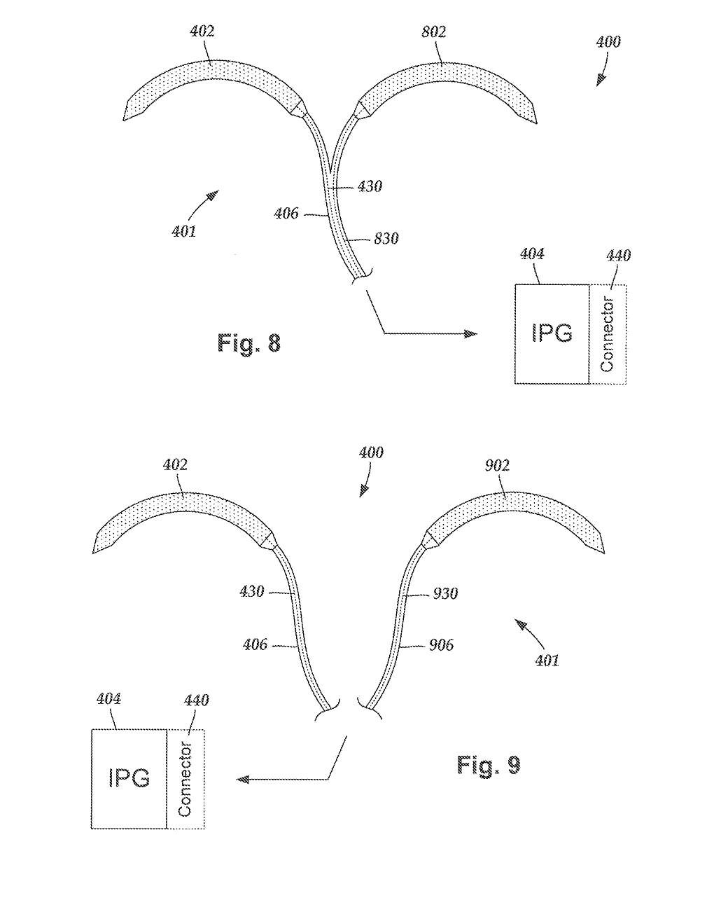

Turning to FIG. 8, in at least some embodiments the lead assembly includes multiple penetrating electrodes coupled to a single tether. FIG. 8 is a schematic view of another embodiment of the lead assembly 401 that includes multiple penetrating electrodes 402 coupled to a single tether 406. In FIG. 8, the system 400 is shown having two penetrating electrodes 402, 802 coupled to the tether 406 which, in turn, is configured and arranged for coupling to the pulse generator 404 or any of the other arrangements illustrated in FIG. 4.

In at least some embodiments, each of the penetrating electrodes 402, 802 is coupled to a different conductor extending along the tether 406. In FIG. 8, the conductor 430 is coupled to the penetrating electrode 402, while the conductor 830 is coupled to the penetrating electrode 802. In at least some embodiments, the conductor 430 and the conductor 830 are each individually encased in conductor insulation, as discussed above with reference to FIG. 4. In at least some embodiments, the conductor 430 and the conductor 830 are collectively encased in tether insulation, as discussed above with reference to FIG. 4.

It may be advantageous to design the lead assembly 401 to include a plurality of penetrating electrodes each coupled to a single tether. Such a configuration enables each of the penetrating electrodes to be individually placed and individually operated with different operational parameters. Additionally, such a configuration adds additional penetrating electrodes without increasing complexity associated with adding additional tethers 406 to the lead assembly 401.

Turning to FIG. 9, in at least some embodiments the lead assembly includes multiple penetrating electrodes coupled to multiple tethers. FIG. 9 is a schematic view of yet another embodiment of the lead assembly 401 that includes multiple electrodes 402, 902 coupled to multiple tethers 406, 906. In FIG. 9, the system 400 is shown having two penetrating electrodes 402, 902, where the penetrating electrode 402 is coupled to the tether 406, while the penetrating electrode 902 is coupled to the tether 906. Each of the tethers 406, 906 is configured and arranged for coupling to the pulse generator 404.

In FIG. 9, a conductor 430 is coupled to the penetrating electrode 402 and extends along the tether 406, while another conductor 930 is coupled to the penetrating electrode 402 and extends along the tether 906. In at least some embodiments, the conductor 430 and the conductor 930 are each individually encased in conductor insulation, as discussed above with reference to FIG. 4. In at least some embodiments, the conductor 430 and the conductor 930 are each collectively encased in tether insulation along their respective tethers, as discussed above with reference to FIG. 4.

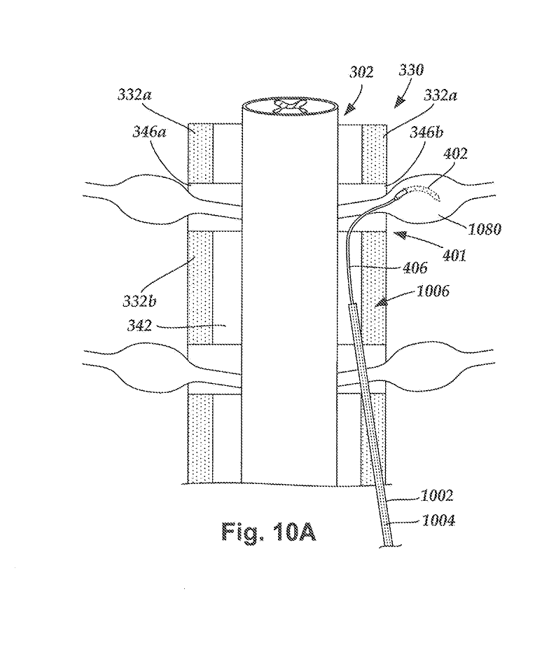

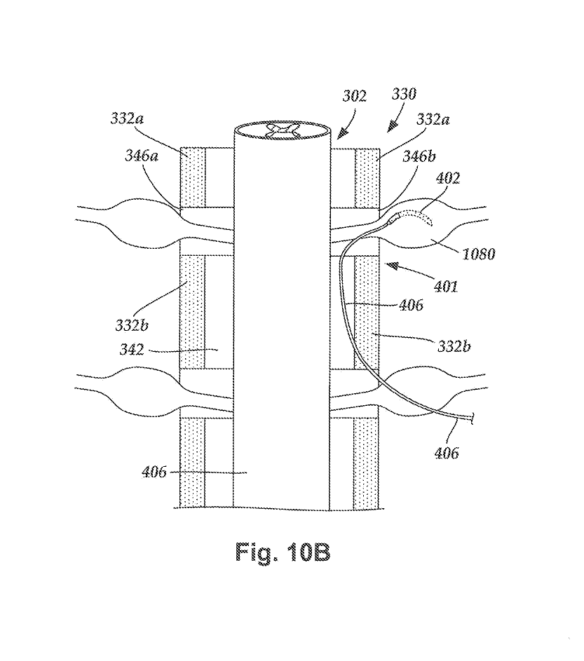

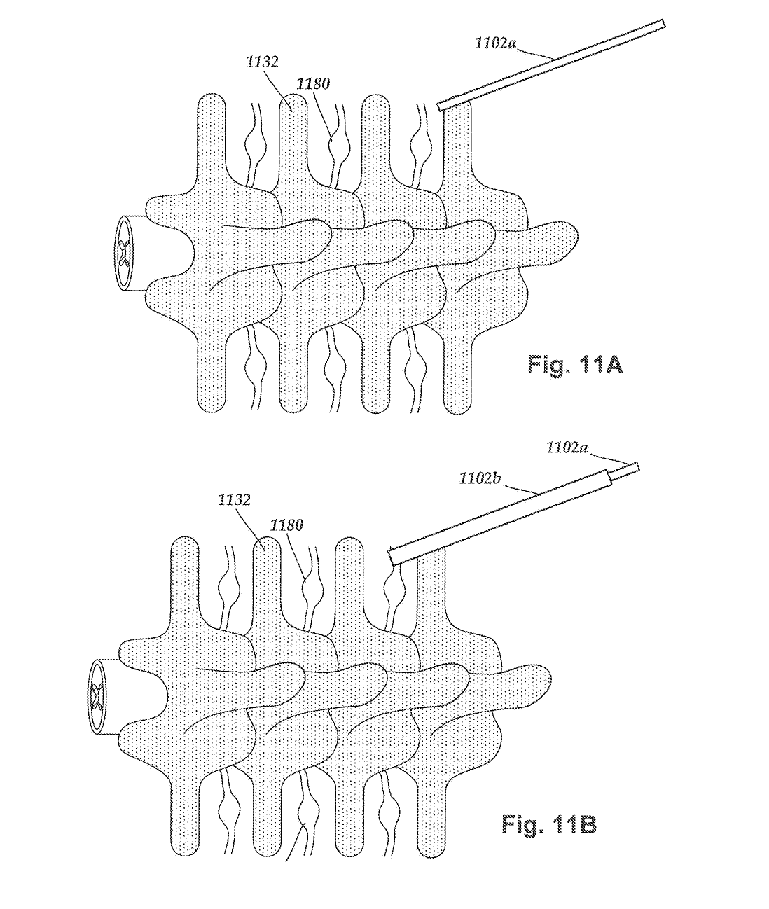

Turning to FIGS. 10A-10B, the lead assembly 401 can be implanted into a patient in any suitable manner. In embodiments where the target stimulation location is one of the patient's DRG, the lead assembly 401 can, optionally, be introduced through the patient's epidural space. Although the DRG are not within the epidural space, one or more of the DRG may be accessible to the lead assembly 401 from within the epidural space via the intervertebral foramina.

In some instances, once the lead assembly 401 is inserted into the epidural space, the one or more penetrating electrodes 402 can be advanced out of the epidural space through one of the intervertebral foramen, and into the desired DRG. In at least some embodiments, a needle is used to introduce the lead assembly into the patient's epidural space, or to the DRG. Alternately or additionally, a sheath may be used to facilitate implantation of the lead assembly 401. Examples of using sheaths to implant a lead assembly can be found in, for example, U.S. patent application Ser. No. 13/900,247, which is incorporated by reference.

FIG. 10A is a schematic perspective view of the spinal cord 302 disposed along a longitudinal transverse view of a portion of the vertebral column 330. The portion of the vertebral column 330 shown in FIG. 10A includes the vertebrae 332a and 332b and intervertebral foramina 346a and 346b defined between the vertebrae 332a and 332b on opposing sides of the vertebral column 330. A DRG 1080 extends outward from one side of the spinal cord 302 and through the intervertebral foramen 346b.

In FIG. 10A, a distal portion 1006 of a needle 1002 is shown disposed in the epidural space 342. The needle 1002 is configured and arranged for introducing the lead assembly 401 into the epidural space 342 through a lumen 1004 defined along a length of the needle 1002. In at least some embodiments, the needle 1002 has a gauge size of at least 18, 19, or 20. In at least some embodiments, the lumen 1004 of the needle 1002 has an inner diameter that is no greater than 0.03 inches (approximately 0.09 cm), 0.02 inches (approximately 0.06 cm), or less. In at least some embodiments, the needle 1002 defines one or more slits defined along a length of the needle 1002 for facilitating removal of the tether 406 when, for example, one or more portions of the lead assembly 401 is larger than the inner diameter of the needle 1002.

In at least some embodiments, once the distal portion 1006 of the needle 1002 is disposed at the target implantation location (e.g., the epidural space 342), the lead assembly 401 is advanced along the lumen 1004 of the needle 1002 to the distal portion 1006 and the needle 1002 is removed from the patient (as shown in FIG. 10B), leaving the penetrating electrode 402 of the lead assembly 401 disposed in the epidural space 342. The penetrating electrode 402 is advanced through the intervertebral foramen 346b and placed in the DRG 1080. In at least some embodiments, a stiffener (e.g., a sheath, stylet, or the like) is used to facilitate advancement of the penetrating electrode 402 to the DRG 1080 from within the epidural space 342.

Alternately, the DRG can be accessed without entry of the lead assembly 401 into the epidural space. In instances where the DRG is accessed without entry of the lead assembly 401 into the epidural space, the lead assembly 401 may extend through a relatively thick area of tissue, including muscle tissue, to implant the lead assembly.

In at least some embodiments, the lead assembly is introduced to the DRG using a set of introducers, each introducer in the set having a larger inner diameter than the preceding introducer. Examples of using a set of introducers to implant a lead assembly can be found in, for example, U.S. Pat. No. 7,993,378; and U.S. patent application Ser. No. 13/900,320, both of which are incorporated by reference.

In at least some embodiments, the introducers can be inserted into the patient sequentially starting with the introducer having the smallest inner diameter. This first introducer of the set may be inserted over a guidewire or over or through a needle that has been inserted into patient tissue. Each subsequent introducer is inserted over the preceding introducer so that the opening into the patient becomes sequentially larger. In at least some embodiments, a larger diameter introducer may be inserted deeper into the tissue than the preceding introducer, thereby increasing the depth of the opening, as well as expanding its size.

Once the set of introducers has been inserted into the patient, one or more of the smaller diameter introducers (and, in at least some embodiments, all of the introducers except the one with the largest diameter) are removed to leave a passage with access to the DRG and sufficient space to permit a practitioner to pierce the DRG with the penetrating electrode 402.

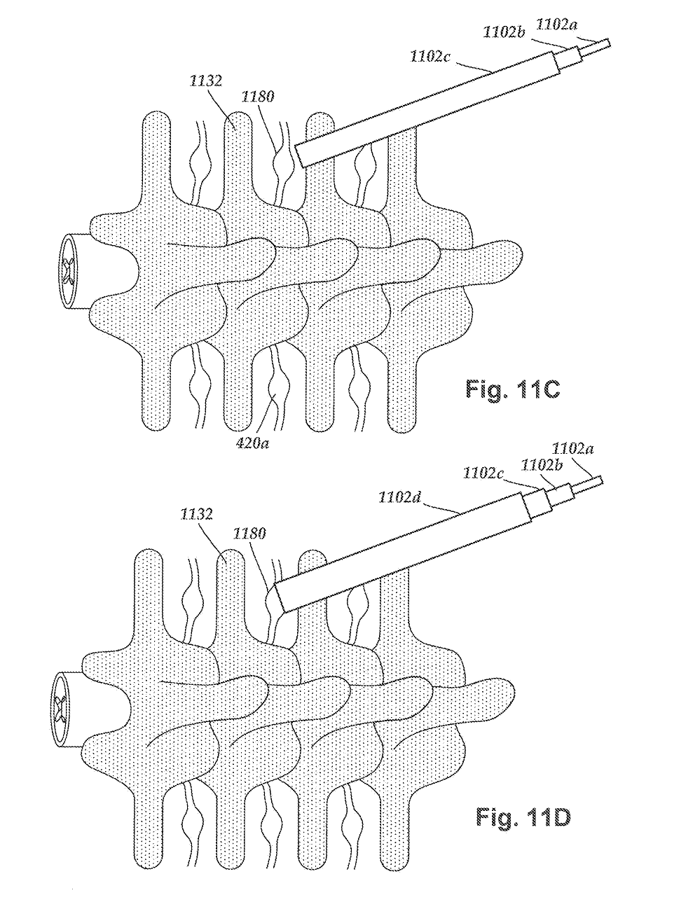

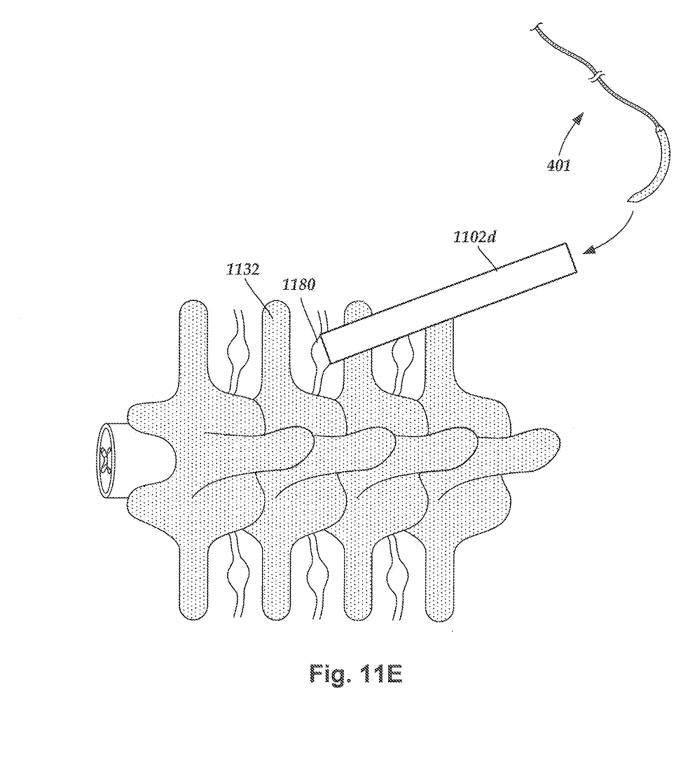

FIGS. 11A-11E schematically illustrate one embodiment of a method of implanting the lead assembly 401 using a series of introducers. FIG. 11A illustrates the insertion of a first introducer 1102a into patient tissue near a vertebra 1132 and directed toward a target DRG 1180. This first introducer 1102a may be directed into the tissue without any guiding element or the first introducer may be inserted over a guidewire, needle, or the like (not shown) that had been previously inserted into the tissue. In at least some embodiments, an obturator or trocar is provided within the first introducer to reduce or prevent coring of tissue during insertion of the first introducer.

FIG. 11B illustrates the insertion of a second introducer 1102b, with a larger inner diameter than the first introducer 1102a, over the first introducer 1102a. FIG. 11C illustrates the insertion of a third introducer 1102c, with a larger inner diameter than the second introducer 1102b, over the second introducer 1102b. FIG. 11D illustrates the insertion of a fourth introducer 1102d, with a larger inner diameter than the third introducer 1102c, over the third introducer 1102c. In the FIGS. 11A-11E, each subsequent introducer is pushed closer to the target DRG 1180. In other embodiments, the introducers may be inserted to identical, or near identical, depth in the vicinity of the target DRG 1180. Any number of introducers can be used including, but not limited to, two, three, four, five, six, eight, or more. Typically, but not necessarily, the inner diameter of an introducer is larger than the outer diameter of the preceding introducer(s). Preferably, the introducers slide over each other. In at least some embodiments, a lubricating substance may be used to facilitate sliding of an introducer over the preceding introducer in the series.

Once the introducers have been inserted, one or more of the earlier introducers are removed. As illustrated in FIG. 11E, the first introducer 1102a, second introducer 1102b, and third introducer 1102c are removed, leaving the fourth introducer 1102d and a passage defined by the fourth introducer and extending from outside the body to the dorsal root ganglion. In some embodiments, one or more of the preceding introducers may be left within the final introducer to, for example, provide structural stability.

Once the passage is open, the lead assembly 401 can then be implanted through the passage defined by the introducer 1102d. The practitioner may select the diameter of the introducer 1102d and the resulting passage to facilitate the implantation of the lead assembly 401. Factors that can affect the diameter of the passage include, but are not limited to, the size of the lead assembly, the desired implantation site, trauma to the tissue through which the introducer passes, and the like.

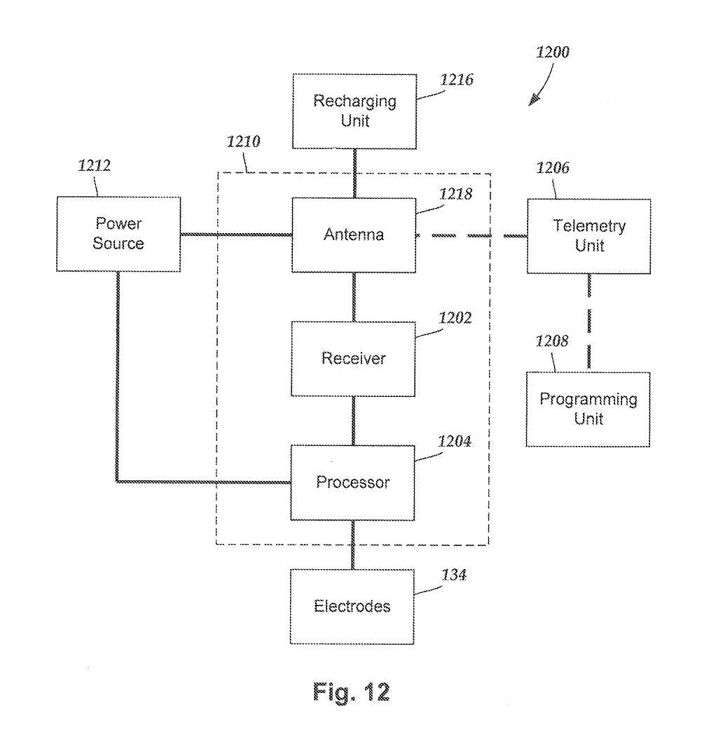

FIG. 12 is a schematic overview of one embodiment of components of an electrical stimulation system 1200 including an electronic subassembly 1210 disposed within a control module. It will be understood that the electrical stimulation system can include more, fewer, or different components and can have a variety of different configurations including those configurations disclosed in the stimulator references cited herein.

Some of the components (for example, power source 1212, antenna 1218, receiver 1202, and processor 1204) of the electrical stimulation system can be positioned on one or more circuit boards or similar carriers within a sealed housing of an implantable pulse generator, if desired. Any power source 1212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1218 or a secondary antenna. The external power source can be in a device that is mounted on the skin of the user or in a unit that is provided near the user on a permanent or periodic basis.

If the power source 1212 is a rechargeable battery, the battery may be recharged using the optional antenna 1218, if desired. Power can be provided to the battery for recharging by inductively coupling the battery through the antenna to a recharging unit 1216 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1204 is generally included to control the timing and electrical characteristics of the electrical stimulation system. For example, the processor 1204 can, if desired, control one or more of the timing, frequency, strength, duration, and waveform of the pulses. In addition, the processor 1204 can select which electrodes can be used to provide stimulation, if desired. In some embodiments, the processor 1204 may select which electrode(s) are cathodes and which electrode(s) are anodes. In some embodiments, the processor 1204 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1208 that, for example, allows modification of pulse characteristics. In the illustrated embodiment, the processor 1204 is coupled to a receiver 1202 which, in turn, is coupled to the optional antenna 1218. This allows the processor 1204 to receive instructions from an external source to, for example, direct the pulse characteristics and the selection of electrodes, if desired.

In one embodiment, the antenna 1218 is capable of receiving signals (e.g., RF signals) from an external telemetry unit 1206 which is programmed by a programming unit 1208. The programming unit 1208 can be external to, or part of, the telemetry unit 1206. The telemetry unit 1206 can be a device that is worn on the skin of the user or can be carried by the user and can have a form similar to a pager, cellular phone, or remote control, if desired. As another alternative, the telemetry unit 1206 may not be worn or carried by the user but may only be available at a home station or at a clinician's office. The programming unit 1208 can be any unit that can provide information to the telemetry unit 1206 for transmission to the electrical stimulation system 1200. The programming unit 1208 can be part of the telemetry unit 1206 or can provide signals or information to the telemetry unit 1206 via a wireless or wired connection. One example of a suitable programming unit is a computer operated by the user or clinician to send signals to the telemetry unit 1206.

The signals sent to the processor 1204 via the antenna 1218 and receiver 1202 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1200 to cease operation, to start operation, to start charging the battery, or to stop charging the battery. In other embodiments, the stimulation system does not include an antenna 1218 or receiver 1202 and the processor 1204 operates as programmed.

Optionally, the electrical stimulation system 1200 may include a transmitter (not shown) coupled to the processor 1204 and the antenna 1218 for transmitting signals back to the telemetry unit 1206 or another unit capable of receiving the signals. For example, the electrical stimulation system 1200 may transmit signals indicating whether the electrical stimulation system 1200 is operating properly or not or indicating when the battery needs to be charged or the level of charge remaining in the battery. The processor 1204 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An implantable lead assembly for providing electrical stimulation to a patient, the lead assembly comprising:
at least one first penetrating electrode configured and arranged for stimulating patient tissue, the at least one first penetrating electrode having an outer surface and comprising at least one sharpened tip configured and arranged to pierce patient tissue and anchor the lead assembly to the pierced patient tissue;
an elongated, flexible first tether having a first end and an opposing second end, the first end coupled to the at least one first penetrating electrode, and the second end configured and arranged to couple to a pulse generator; and
at least one elongated first conductor electrically coupled to the at least one first penetrating electrode and extending therefrom along the first tether.

2. The lead assembly of claim 1, further comprising an implantable lead body with a proximal end, a distal end, and a longitudinal length, wherein the lead body comprises
at least one terminal that is disposed along the proximal end of the lead body and that is electrically coupled to the at least one first conductor, wherein the at least one terminal is configured and arranged to couple with the pulse generator, and
an aperture defined along the distal end of the lead body, the aperture configured and arranged for the first tether to extend through the aperture.

3. The lead assembly of claim 1, further comprising a strain relief coupleable to the tether, the strain relief configured and arranged to reduce the likelihood of decoupling between the lead assembly and at least one of the pulse generator or patient tissue during patient movement.

4. The lead assembly of claim 1, wherein the at least one first penetrating electrode further comprises a first curved region.

5. The lead assembly of claim 4, wherein the at least one first penetrating electrode further comprises a second curved region.

6. The lead assembly of claim 1, wherein the at least one sharpened tip of the at least one first penetrating electrode comprises a plurality of sharpened tips each configured and arranged to pierce patient tissue.

7. The lead assembly of claim 1, wherein the at least one first electrode comprises a plurality of first electrodes, each of the plurality of first electrodes coupled to the first tether.

8. The lead assembly of claim 7, wherein the at least one first conductor comprises a plurality of first conductors, each of the plurality of first conductors coupled to a different first electrode of the plurality of first electrodes.

9. The lead assembly of claim 1, wherein the lead assembly further comprises at least one second penetrating electrode configured and arranged for stimulating patient tissue, the at least one second penetrating electrode comprising at least one sharpened tip configured and arranged to pierce patient tissue.

10. The lead assembly of claim 9, wherein the lead assembly further comprises:
an elongated, flexible second tether having a first end and an opposing second end, the first end of the second tether coupled to the at least one second penetrating electrode; and
at least one elongated second conductor electrically coupled to the at least one second penetrating electrode and extending therefrom along the second tether.

11. The lead assembly of claim 1, wherein the sharpened portion of the at least one first electrode is configured and arranged to pierce a dorsal root ganglion of the patient.

12. The lead assembly of claim 1, wherein the first tether has a diameter that is no greater than 0.08 cm.

13. The lead assembly of claim 1, wherein the outer surface of the at least one first electrode comprises at least one of a notch or a knurl for increasing the surface area of the at least one first electrode.

14. The lead assembly of claim 1, further comprising a coating disposed over the outer surface of the at least one first electrode, the coating configured and arranged for increasing the area of the outer surface of the least one first electrode.

15. An electrical stimulation system comprising:
the lead assembly of claim 1; and
a pulse generator coupleable to the second end of the first tether of the lead assembly, the pulse generator also coupleable to the at least one first conductor extending along the first tether.

16. The electrical stimulation system of claim 15, wherein the pulse generator is disposed in an implantable control module, the control module comprising
a housing, and
an electronic subassembly disposed in the housing, wherein the pulse generator is disposed in the electronic subassembly.

17. A method for implanting a lead assembly of an electrical stimulation system into a patient, the method comprising:
advancing the lead assembly of claim 1 into the patient with the at least one first penetrating electrode of the lead assembly in proximity to one of the patient's dorsal root ganglia; and
piercing the dorsal root ganglion with the at least one sharpened tip of the at least one first penetrating electrode to anchor the at least one first penetrating electrode to the dorsal root ganglion.

18. The method of claim 17, wherein advancing the lead assembly of claim 1 into the patient with the at least one first penetrating electrode of the lead assembly in proximity to one of the patient's dorsal root ganglia comprises advancing the at least one first penetrating electrode through a portion of the patient's epidural space.

19. The method of claim 17, wherein advancing the lead assembly of claim 1 into the patient with the at least one first penetrating electrode of the lead assembly in proximity to one of the patient's dorsal root ganglia comprises extending the lead assembly through a lumen of a needle, wherein the lumen of the needle has an inner diameter that is no greater than 0.09 cm.

20. The method of claim 17, wherein advancing the lead assembly of claim 1 into the patient with the at least one first penetrating electrode of the lead assembly in proximity to one of the patient's dorsal root ganglia comprises advancing the at least one first penetrating electrode using a set of introducers.
